(19) United States
(12) Patent Application Publication
(10) Pub. No.: US 2009/0023175 A1
(43) Pub. Date: Jan. 22, 2009

(54) Device for sampling a fluid and detecting an analyte therein

(76) Inventors: Remco Maria Es, Gissenburg (NL); Tim de Graaf, Zoetermeer (NL); Marinus Hubertus Nicolaas Berg, Maastricht (NL); Frederic William Clifton Tassie, Nieuwstadt (NL)

Correspondence Address: NIXON & VANDERHYE, PC, 901 NORTH GLEBE ROAD, 11TH FLOOR, ARLINGTON, VA, 22203, US

(21) Appl. No.: 11/665,004

(22) PCT Filed: Oct. 20, 2005
(86) PCT No.: PCT/EP2005/055426

§ 371 Date: Apr. 10, 2007

(30) Foreign Application Priority Data
Oct. 20, 2004 (EP) 04105186.3
Apr. 5, 2005 (EP) 0512674.8

(51) Int. Cl. C12Q 1/02; B01J 19/00; C12M 1/00

(52) U.S. Cl. 435/29; 422/68.1; 435/287.1

(57) ABSTRACT

Provided is a device for detecting an analyte in a fluid comprising a first compartment (1) having an open end (1.2) and a closed end (1.1) wherein said closed end holds a medium (1.3) comprising a substance with which said analyte can be detected and a second compartment (2) having two open ends (2.1, 2.2) wherein the one open end (2.1) of the second compartment is connectable to the open end (1.2) of the first compartment and the other open end of the second compartment has a means (2.3.2) for withdrawing a predetermined volume of said fluid.

### FIELD OF THE INVENTION

The present invention relates to a device for sampling a fluid and detecting an analyte therein.

### BACKGROUND OF THE INVENTION

Devices suitable for detecting analytes in samples are known for many years in various applications. For instance, microbiological test methods for the determination of antibacterial compounds, particularly residues of antibiotics such as cephalosporin, penicillin, tetracycline and derivatives thereof and chemotherapeutics such as sulfadiazines and similar compounds (sulfa's), in fluids such as milk, meat juice, serum and urine have been described in CA 2056581, DE 3613794, EP 0005891, EP 0285792, EP 0611001, GB A 1467439 and U.S. Pat. No. 4,946,777. These descriptions all deal with ready to use tests that make use of a test microorganism and will give a result by the change indicated by an indicator molecule, for instance a change of color of a pH- and/or redox-indicator. The test systems described above usually include a test medium, such as agar in which test components are present. Such test components may be a test microorganism, indicator molecules and/or nutrients, but also other components are conceivable to the person skilled in the art. In most examples of test systems known today, the test medium is contained within a container such as a tube or a well in a microtiter plate. The sample to be analyzed then needs to be brought into said container onto the test medium. Usually, said containers are sealed in order to prevent contamination and in some cases auxiliary containers with required components such as nutrients are supplied from which said required components are to be transferred to the container with the test medium.

The problem with the test systems currently distributed on the market and/or described in literature is that they require multiple operations to be performed by the user. Since any operation is subject to the occurrence of errors, a high number of operations gives rise to a high number of possible errors and thus to a relatively unreliable and inconvenient test system. For example, a given test system may require operations such as opening an ampoule, addition of nutrients, attachment of a pipette to a syringe, withdrawal of a sample with the pipette/syringe, and application of the sample to the test medium. In most cases many or all these operations need to be performed under aseptic conditions. Yet another problem associated with the test systems currently distributed on the market and/or described in literature is that unwanted cross-contamination can easily occur.

Reducing the risk for errors in test systems by simplification of the equipment is a problem that has been addressed before. For instance, EP 0271102 describes a sample collection device wherein a liquid-absorbing material is used to absorb the required amount of sample without having to use complicated equipment. However, said improved test systems and methods still require multiple devices and process steps, each of which introduce risks for errors. Thus, there is still a need for an improved test system that does not have these problems.

### SUMMARY OF THE INVENTION

It is an object of the present invention to provide a device and an improved method for the determination of analytes in fluids. The device of the present invention allows for such determination with an ease of handling that is unprecedented. The present invention provides a device for detecting an analyte in a fluid comprising a compartment (0) having a closed end (1.1) and an open end (2.2), wherein a test medium (1.3) comprising a substance with which said analyte can be detected is present in said compartment (0) at said closed end (1.1) and wherein said open end (2.2) has a means (2.3) for withdrawing a predetermined volume of said fluid.

Furthermore, the present invention provides a method for determining the presence or absence of an analyte in a fluid comprising:
(a) inserting the open end (2.2) of said compartment (0) of the device into said fluid;
(b) optionally applying external pressure on said compartment (0) followed by release of pressure;
(c) withdrawing said device from said fluid;
(d) optionally removing excess fluid from the interior of said device until the required volume is obtained;
(e) turning said device such that the fluid remaining in said device is contacted with a test medium (1.3) suitable for detecting said analyte.

Also, the present invention provides the use of a device as described above for sampling and analyzing a fluid.

### DETAILED DESCRIPTION OF THE INVENTION

The terms and abbreviations given below are used throughout this disclosure and are defined as follows.

The term ‘CFU’ is an abbreviation of Colony Forming Units and refers to the number of microorganisms spores of microorganisms, partially germinated spores of microorganisms, vegetative cells or spores of vegetative cells capable of producing colonies of microorganisms.

The term ‘drip ring’ refers to a means attached to the exterior of a device and fully enclosing the device which is designed such that any fluid which is present on the outside of the device is contained within the drip ring when the device is turned upside down. When the device is cylindrical, the drip ring is also cylindrical. Likewise, when the device has any other form, the drip ring has the same form as the device.

The term ‘fluid’ refers to a substance (as a liquid) tending to flow or conform to the outline of its container.

The term ‘gelling agent’ refers to a compound that assists in changing a mixture into or taking on the form of a gel.

The term ‘indicator’ refers to a substance used to show (for example by change of color or fluorescence) the condition of a mixture such as a solution or a gel with respect to the presence of a particular material (for example an acid, a base, oxidizing or reducing agents). For instance, the term ‘indicator’ may refer to one or more compounds that are known as pH-indicators, but also to one or more compounds that are known as redox-indicators. Also, the term ‘indicator’ may refer to mixtures of two or more different types of indicators, such as a combination of a pH- and a redox-indicator. In general, when two or more indicators are used, these indicators are co-operating to increase the indicator effect of each of the indicators when taken alone.

The term ‘nutrient’ refers to one or more nutritive substances or ingredients that promote and/or are required for the growth of microorganisms as used in an embodiment of the present invention.

The term ‘sampling device’ refers to a device with the aid of which a sample of a fluid can be added to a test medium. Such a device may be a container, optionally with volume markings. Such a container may be a capillary, a syringe, a pipette or an automated pipetting system. Such a syringe or pipette may be designed in such a fashion that with only one mode of operation a predetermined volume can be withdrawn from the fluid to be analyzed. Alternatively, a sampling device is a device suitable for obtaining fluid samples from solid materials such as meat. In the context of the present invention, the container of the sampling device may be equipped with cutting edges that are suitable for removing solid samples that can subsequently be pressed in the sampling device until a fluid sample is obtained.

The term ‘spore’ refers to a primitive usually unicellular often environmentally resistant dormant or reproductive body produced by plants or microorganisms and capable of development into such a plant or microorganism.

The term ‘test medium’ refers to a solid composition, preferably in the form of a sol or a gel, for instance comprising a gelling agent. Suitable examples of gelling agents are agar, alginic acid and salts thereof, carrageenan, gelatin, hydroxypropylguar and derivatives thereof, locust bean gum (Carob gum), processed eucheuma seaweed and the like. However, the person skilled in the art will understand that other types of solid test media may be based on carrier materials such as ceramics, cotton, glass, metal particles, paper, polymers in any shape or form, silicates, sponges, wool and the like. Usually, a test medium contains one or more indicators, however, these compounds may also be added during the test method. The test medium comprises one or more types of test microorganisms as detecting agents. Optionally, the test medium may also contain nutrients, stabilizers, and substances that change the sensitivity to certain antimicrobial compounds in a positive or negative way, and/or viscosity-increasing agents. Examples of substances that change the sensitivity to certain antimicrobial compounds are antifolates like ormethoprim, tetroxoprim and trimethoprim that improve the sensitivity of the test organism towards sulfa compounds or salts of oxalic acid or hydrofluoric acid, which improve the sensitivity towards tetracycline. Examples of viscosity-increasing agents are ascorbyl methylsilanol pectinate, carbomer, carboxymethyl cellulose, cetearyl alcohol, cetyl alcohol, cetyl esters, cocamide DEA, emulsifying wax, glucose, hydroxyethyl cellulose, hydroxypropylmethyl cellulose, lauramide DEA, linoleamide DEA, magnesium aluminum silicate, maltodextrins, PEG-8 distearate, polyacrylamide, polyvinyl alcohol, PVP/hexadecene copolymer, sodium chloride, sodium sulfate, soyamidopropyl betaine, xanthan gum and the like. Alternatively, the optional ingredients of the test medium mentioned above may also be added exogenously.

The term ‘threshold’ refers to the concentration value above which a given analyte is to be regarded as present and below which said analyte is to be regarded as absent. Generally, a threshold value is given for particular analytes in particular samples by local, regional or interregional authorities but it can also be pre-set for certain research purposes.

In a first aspect of the invention, there is provided a device, an example of which is given in FIG. 1, comprising a first compartment (1) having a closed end (1.1) and an open end (1.2) wherein said closed end is suitable for holding a medium (1.3) comprising a substance with which an analyte can be detected, and a second compartment (2) having two open ends wherein the one open end (2.1) of the second compartment is connectable to the open end of the first compartment and the other open end (2.2) of the second compartment has a means for withdrawing a predetermined volume of said fluid. In this respect, being connectable refers to the fact that two open ends of the first and second compartments can be connected to one another, i.e. by means of a separate member, but may also be connected directly, for instance during production by welding the two compartments together using any type of glue or the same material as is used for the production of the compartments.

Said other open end (2.2) of said second compartment (2) is optionally sealed with a cap (5) such as a screw cap, a breakable cap, a filmhinge or any other suitable sealing means. Preferably, the diameter of said other open end (2.2) is such that evaporation of sample and/or medium components is kept to a minimum. In many devices known in the art said evaporation leads to loss in accuracy of the test system. Suitable diameters of open end (2.2) in this respect are from 0.01-2 cm, preferably from 0.05-1 cm, more preferably from 0.1-0.5 cm, most preferably from 0.2-0.3 cm. Although different shapes are conceivable and by no means unsuited for the present invention, the usual shape of the first and second compartments is cylindrical with diameters ranging from 0.2-10 cm, preferably from 0.4-5 cm, more preferably from 0.6-2 cm, most preferably from 0.9-1.4 cm and lengths ranging from 0.5-20 cm, preferably from 1-10 cm, more preferably from 2-8 cm, most preferably from 3-5 cm. Normally the ratio between length and diameter is from 0.1-100, preferably from 1-50, more preferably from 2-20, most preferably from 5-10.

Suitable materials of which the device of the present invention may be constructed are plastics such as polyacrylics, polycarbonates, polyethylene, polyvinyl chloride and the like. The devices of the first aspect of the present invention can all be easily made using standard protocols known to the person skilled in the art. Traditional and well-known processes such as injection molding and blow-molding can be used for the production of all devices of the present invention. Production of these devices can be performed with many types of materials using standard molding equipment, which can be adapted to the required dimensions. Introduction of the test medium into the devices of the present invention can be performed in several ways. One option, as outlined in FIG. 7, comprises the separate production of the first and second compartments, followed by insertion of the test medium in the first compartment, followed by attaching the first and second compartments to one another. This construction method can be applied to all devices of the present invention when constructed from two separate compartments. In another option, as outlined in FIG. 10, the device is produced in one piece by e.g. blow-molding or injection molding followed by introduction of the test medium through open end 2.2. This construction method can be applied to all devices of the present invention when constructed in one piece. Most preferable, the test medium is introduced in the device in liquid form, i.e. at elevated temperatures (e.g. ranging from 70 to 150° C.), whereupon the test medium is allowed to solidify, i.e. by cooling to lower temperatures such as ranging from −10 to 50° C. Other methods of obtaining this liquid to solid conversion, i.e. photochemical conversions, irradiation and other chemical conversion may also be employed.

Preferably the fluid that is to be withdrawn is a fluid that may or may not contain one or more analytes that are to be detected. Examples of such fluids are beverages, blood, cream, eggs, fruit juices, honey, meat juice, milk, milk products, urine and the like. The volume of fluid that is to be withdrawn usually ranges from 0.01-10 ml, preferably from 0.05-5 ml, more preferably from 0.1-1 ml, most preferably from 0.2-0.5 ml. Examples of analytes that are to be detected are antibiotics, carbohydrates, hormones, metals, microorganisms, nucleic acids, peptides, salts, toxic components and the like.

In an embodiment of the first aspect of the invention, the device allows for the incorporation of a medium comprising a substance with which an analyte can be detected. A device of this type has the advantage that sample withdrawal and measurement can be carried out in one and the same device as the sample analysis. This reduces the amount of operations to be performed by the user, and consequently reduces the occurrence of errors and increases the reliability. Thus there is provided a device that is partially filled with a test medium such as carrier material and/or a test medium comprising a substance with which an analyte can be detected in a fluid. The person skilled in the art will appreciate that the device of the second aspect may be filled with a wide variety of media that are suitable for detecting various analytes. Such a test medium may contain an indicator or may be an indicator itself. Preferably, the test medium is solid which has the advantage that the test medium stays in place when the device is rotated.

In a second embodiment, the device may be partially filled with a liquid such as a solution comprising a substance with which an analyte can be detected in a fluid. In order to prevent loss of this liquid, the closed end of the first compartment comprises an insert that is constructed such that said liquid cannot leave the device when the device is turned in order to withdraw sample fluid.

The test medium may be any medium suitable for the detection of an analyte in a fluid. It may be a medium comprising or consisting of an indicator. For the detection of antibiotics in fluids, for instance, the test medium or the liquid may also comprise a test organism, nutrients for the test organism, a substance that provides a solid state and at least one indicator. Suitable nutrients in this respect are carbon-sources and nitrogen-sources of which many commercially available variants exist. Typical constituents are amino acids, monosaccharides, vitamins and the like. Also oligosaccharides may be present as nutrient. Preferably the oligosaccharide is partly soluble in aqueous solutions and preferably the oligosaccharide contains one or more glucose units. More preferably the oligosaccharide is a relatively short-chained oligosaccharide such as a disaccharide, a trisaccharide, a tetrasaccharide or a pentasaccharide. Most preferably the oligosaccharide is a disaccharide such as cellobiose, gentiobiose, lactose, maltose, sucrose or trehalose. Suitable trisaccharides are maltotriose, melezitose and raffinose.

The person skilled in the art is well aware of methods available for the preparation of suitable test media. An example is given in U.S. Pat. No. 4,946,777. For reasons of clarity, the method described herein, which is only one of many examples, is repeated below.

A culture of Bacillus stearothermophilus var. calidolactis L.M.D. 74.1 was inoculated on a medium consisting of:
Bacto nutrient agar, Difco code 0001 (15 g)
Bacto agar, Difco code 0140 (5 g)
dextrose (0.5 g)
MnSO₄.H₂O (30 mg)
distilled water to 1000 ml,

which was sterilized for 20 minutes at 120° C.

After inoculation, the medium was incubated at 60° C. for at least 48 hours until a good sporulation was observed. The spores were then collected, washed with distilled water and stored at 4° C. The amount of viable spores was detected by testing on a medium consisting of:
Bacto agar, Difco code 0140 (20 g)
Bacto Tryptone, Difco code 0123 (8.5 g)
Phytone Peptone, BBL code 11905 (1.5 g)
dextrose (5 g)
distilled water to 1000 ml,

which was sterilized for 20 minutes at 120° C.

After inoculation, the medium was incubated for 48 hours at 60° C. after which the colonies were counted. Distilled water was added to, or water was removed from, the spore suspension until the suspension contained about 10⁸ viable germs per ml. One percent of the above-mentioned spore suspension containing 10⁸ germs per ml was added to the following solution:
Bacto agar, Difco code 0140 (12 g)
sodium chloride (9 g)
distilled water to (1000 ml), which was sterilized for 20 minutes at 120° C.

The medium was liquefied by heating and then was cooled at 60° C. Devices of the dimensions of the present invention were each filled with 0.5 ml of the thus obtained medium and the contents of the devices were allowed to solidify with the devices being held in an upright position. The devices were stored at a temperature of 4° C.

In U.S. Pat. No. 4,946,777, there is also a description for the preparation of so-called Nutrient Discs, the purpose of which is to be added to the test medium upon start of the test (as i.e. envisioned with the additional compartment (3) of FIG. 2). Not only can this preparation be used for Nutrient Discs, the respective contents can also be added to the test medium as described above, as the skilled person can easily perform. Again the preparation of the Nutrient Discs is repeated for reasons of clarity:
(a) Bromocresol purple (0.1 g) dissolved in 9.2 ml of 0.02 N NaOH was diluted with distilled water to make 25 ml.
(b) dextrose 50 g
   distilled water 50 ml
(c) Bacto Tryptone, Difco code 0123 (34 g)
   Phytone Peptone, BBL code 11905 (6 g)
   distilled water 100 ml

Solutions (a) and (b) were sterilized by passage through a Seitz filter and medium (c) was sterilized at 110° C. for 30 minutes and medium (c) remained a suspension. Five parts of solution (a), two parts of solution (b) and three parts of suspension (c) were mixed together, and 0.04 ml of the solution obtained was contacted with filter paper discs having a cross-sectional dimension of about 8 mm, and the discs were then dried.

The skilled artisan will appreciate that not only Bromocresol Purple, as mentioned above, but also indeed many indicators are suitable for the purpose of the first aspect of the present invention. Particularly useful are indicators that, upon changing from one state to the other, provide a visually detectable signal such as a change in color or fluorescence. The amount of indicator in the test medium is between 0.01 and 50 g.l⁻¹ test medium, preferably between 0.1 and 10 g.l⁻¹, more preferably between 0.5 and 5 g.l⁻¹, most preferably between 1 and 3 g.l⁻¹. Such indicators may be selected from handbooks such as ‘H.J. Conn's Biological Stains’, R. D. Lillie ed., Baltimore, 1969. Preferred indicators are pH-indicators and/or redox indicators. Examples of suitable indicators are Acid Blue 120, Acid Orange 51, Acid Yellow 38, Alizarin acid, Alizarin Blue, Azure A, Azure B, Basic Blue 3, Brilliant Black, Brilliant Cresyl Blue, Brilliant Crocein MOO, Brilliant Yellow, Bromocresol Green, Bromocresol Purple, Bromophenol Blue, Bromophenol Red, Bromothymol Blue, Chlorocresol Green, Congo Red, m-Cresol Purple, Gallocyanine, Indigo Carmine, Janus Green B, Litmus, Methylene Blue, Nile Blue A, Nitrazol Yellow (also referred to as Nitrazine Yellow), o-Nitrophenol, p-Nitrophenol, 1-10 Phenanthroline, Phenolphthalein, Safranine O, Thionin, Thymol Blue, Toluidine Blue and Xylenol Blue.

Preferably, the substance providing for a solid state is a gelling agent and/or a carrier material. The amount of gelling agent in the test medium is between 1 and 200 g.l⁻¹ test medium, preferably between 2 and 50 g.l⁻¹, more preferably between 5 and 20 g.l⁻¹, most preferably between 7 and 15 g.l⁻¹. Preferred gelling agents are agar and gelatin.

The test organism preferably is a thermophilic test organism such as a Bacillus species, preferably Bacillus stearothermophilus, an Escherichia coli species, or a Streptococcus species, preferably Streptococcus thermophilus. These species may be introduced in the test as units capable of producing colonies, or Colony Forming Units (CFU's). Said CFU's may be spores, vegetative cells or a mixture of both. The concentration of said CFU's is expressed as Colony Forming Units per ml of test medium (CFU.ml⁻¹) and is usually in the range of 1×10⁵ to 1×10¹² CFU.ml⁻¹, preferably 1×10⁶ to 1×10¹⁰ CFU.ml⁻¹, more preferably 2×10⁶ to 1×10⁹ CFU.ml⁻¹, most preferably 5×10⁶ to 1×10⁸ CFU.ml⁻¹, or still more preferably 5×10⁶ to 2×10⁷ CFU.ml⁻¹.

In a third embodiment of the first aspect of the invention, the first and second compartments are inseparable and form one compartment (0) having a closed end (1.1) and an open end (2.2).

In a fourth embodiment of the first aspect of the invention, said means for withdrawing a predetermined amount of fluid is an insert (2.3.2 in FIG. 1A) in the second compartment that creates a space (2.3.1) with a predetermined volume. Preferably said insert is shaped in such a fashion that, when the device is placed with said other open end of the second compartment in a fluid, said fluid enters the device and when the device is removed from the fluid said space in said second compartment retains an amount of fluid equal to the volume of said space. The device may be constructed such that the above phenomenon can occur upon insertion of the device in the fluid followed by removal from the fluid. Alternatively, the device may be constructed such that fluid can be sucked in by the creation of a pressure drop and pushed out by the creation of over-pressure. In order to achieve this, at least part of the device is constructed of flexible material. Preferably, the shape of said material can be changed by applying pressure. Said pressure should be above atmospheric pressure and preferably said pressure should be within the range easily achievable by the human hand, i.e. ranging from 0.1-10 MPa, preferably ranging from 0.15-5 MPa, more preferably from 0.2-1 MPa.

In a fifth embodiment of the first aspect of the invention, said means for withdrawing a predetermined amount of fluid is an array of at least one volume marking on said second compartment (2.3.3 in FIG. 1B). In order to determine the amount of fluid present in the device, said at least one volume marking and the contents of the device are visible from the outside. Preferably, the device is constructed from a material with sufficient transparency. The device may be constructed such that fluid can be sucked in by the creation of a pressure drop and pushed out by the creation of over-pressure as outlined in the first embodiment.

In a sixth embodiment, an example of which is given in FIG. 2, there is provided a device comprising a first compartment and a second compartment as described above and a third compartment (3) whereby the separation (3.1) between the first and/or second compartment and the third compartment can be broken by the application of pressure. Said pressure should be above atmospheric pressure and preferably said pressure should be within the range easily achievable by the human hand, i.e. ranging from 0.15-10 MPa, preferably ranging from 0.2-5 MPa, more preferably from 0.3-1 MPa. The third compartment can be used to store components such as nutrients and/or test organisms and/or indicator compounds and/or (bio) catalysts and the like. Some applications may require the addition of components only shortly before or even after introduction of the fluid to the test medium. In these cases, use of the third compartment for storage of said components proved to be useful. Advantageously said components are in the form of a tablet so that they can be easily pushed through the wall separating the first and the second compartments. The requirements with respect to means for withdrawing a predetermined volume of fluid are the same as described above for the first aspect of the invention.

In a seventh embodiment, the first and second compartments are connected through a foldable member as exemplified in FIGS. 3 and 4. The advantage of this embodiment is that the first compartment can be easily filled with medium after which it can be sealed. Upon removal of the seal, folding the foldable member (4.1) joins the open ends of the two compartments. As exemplified in FIG. 5, several devices according to this embodiment may be attached to one another using a member (4.1) that allows for easy removal of individual devices by means of, for instance, a perforation line (4.1.4).

In an eighth embodiment, the device of the first aspect of the invention may be constructed such that fluid samples can be obtained from solids. For instance, this may be achieved by supplying cutting edges at said other open end of said second compartment by means of which a sample can be cut from solid material such as meat or fish or fruit. The part of the device in which said sample will be present is made from flexible material so that fluid can be withdrawn from said sample by the application of pressure.

The devices of the first aspect of the present invention are illustrated in FIGS. 1-11. The illustrations given in these Figures are not limiting with regard to the shape and size of the device of the present invention. For instance, in FIG. 1 the second compartment (2) is depicted as a cylinder ending in a conical shape. However other forms, such as rectangular and/or elliptic forms, are also possible. Likewise, the shape and the ratio of the radii of the compartments in FIGS. 2-11 are not limited to those depicted. The radius of the first compartment (1) may be smaller or larger than that of the second compartment (2). Other deviations are also meant to be included in the invention. For reasons of convenience, first compartment (1) may be formed in such a way that there are markings and/or shape distortions that facilitate the application of pressure.

In a second aspect of the invention, there is provided a method for determining the presence or absence of an analyte in a fluid comprising the steps of inserting the open end of the second compartment of the device of the first aspect of the invention into said fluid, withdrawing a fluid sample and contacting said fluid sample with a test medium comprising a substance with which an analyte can be detected.

In a first embodiment, for the detection of antibiotics, the test medium comprises CFU's of a microorganism and at least one indicator and optionally nutrients for the microorganism. Preferably, the test medium is a sol or gel comprising a gelling agent and/or a carrier material. Advantageously, the method also provides for conditions whereby there is minimal growth of a microorganism prior to the addition of fluid sample. Such conditions comprise an unfavorable temperature and/or an unfavorable pH-value and/or the absence of nutrients essential for growth, provided these conditions do not cause irreversible damage to all CFU's present. After addition of the fluid sample, growth of the microorganism is allowed to take place during a period sufficiently long for the microorganisms to grow in case no antibiotic is present. Growth is encouraged by adding nutrients, optionally before the contacting of said fluid sample, and/or raising the temperature, and/or providing for a pH-value at which the microorganism is able to grow. Alternatively, these conditions may be established prior to contact of the fluid sample with the test medium. Growth of the microorganism is detected by observing the presence or absence of a change of the indicator.

In a second embodiment of the second aspect of the present invention, the antibiotic is β-lactam antibiotic such as a cephalosporin or a penicillin derivative. Examples of such derivatives are amoxicillin, ampicillin, cefadroxil, cefradine, ceftiofur, cephalexin, penicillin G, penicillin V and ticarcillin, but of course many other similar β-lactam derivatives are known and applicable in the method of the present invention.

In a third embodiment of the second aspect of the invention, the test micro-organism is incubated for a predetermined period, preferably within a time span of 0.5 to 4 hours, more preferably between 0.75 to 3 hours, most preferably between 1.0 to 2.75 hours. Preferably the microorganism is incubated at a predetermined temperature, preferably the optimal growth temperature of the microorganism. When, for example, thermo stable microorganisms are used, said temperature is preferably between 40 and 70° C., more preferably between 50 and 65° C., most preferably between 60 and 64° C. Optionally said reaction could be carried out with the aid of a thermostatic device. Alternatively, the time required for growth of the microorganism is equal to the time that is required for a calibration sample with a known amount of analyte(s) to induce a change in the indicator. The latter change occurs when the concentration of analyte in the sample is below the threshold value.

In a fourth embodiment of the second aspect of the present invention, nutrients are added as a separate source, e.g. as a tablet, disc or a paper filter. Also other compounds such as the indicator(s), stabilizers and/or antifolates may be added as a separate source, or optionally incorporated in the nutrient medium.

The presence or absence of an antibiotic is determined by the presence or absence of a change of the indicator or indicators used. When, for example such a change is a color change, said color change may be observed visually. However in one embodiment of the invention said color change is determined using an arrangement that generates digital image data or an arrangement that generates analog image data and converts said analog image data into digital image data followed by interpretation of said digital image data by a computer processor. Such an arrangement, which may for instance be a sample-reading device such as a scanner coupled to a personal computer, is described in International Patent Application WO 03/033728, incorporated by reference, and briefly summarized below.

The arrangement can be used for detecting residues of antibiotics in milk. The commercially available Delvotest® and BR®-test are commonly used. Delvotest® comprises an agar matrix, CFU's of an acid forming microorganism, as well as a color indicator. With the arrangement mentioned above it is possible to automatically scan the bottom side of each of the samples in a test plate. The color and the brightness of the reflected light are registered in three variables, each describing one color component, for instance the so-called L*a*b* model. In the L*a*b* model, the color spectrum is divided in a two-dimensional matrix. The position of a color in this matrix is registered by means of the two variables “a” and “b”. The variable L indicates the intensity (for instance, from light blue to dark-blue). It is possible to make a criterion comprising the a-value, b-value and L-value to make a composite function as follows:

Z=w_{L}.L+wₐ.a+w_{b}.b

where w_{L}, wₐ and w_{b} are weighting factors for the L-value, a-value and b-value, respectively. The values of these weighting factors can be calculated by means of “discriminent analysis”, such that the group mean shows a maximum distance in relation to the spreading. By combining two or more of the color components in the L*a*b* model in a predetermined manner that depends on the type of residue and the sample, an accurate detection is possible. In practice, a certain value of Z at which a test should switch between positive and negative result (the threshold value) can be experimentally predetermined.

In a third aspect of the invention there is provided a kit for carrying out the method of the present invention. Such a kit comprises one or more devices according to the first aspect of the invention filled with test medium. The devices may be of any shape and size and of any material available, provided that observation of indicator changes is possible.

Optionally, said kit comprises a means for sealing of said devices filled with test medium during incubation and/or an insert with instructions for use and/or a means for setting the time needed for incubation.

In a first embodiment of the third aspect of the invention, said kit also comprises nutrients incorporated in the second compartment of the device of the first aspect of the invention. Preferably said nutrients are contained within a medium such as a tablet, disc or a paper filter. The amounts can be predetermined so as to avoid errors in dosing the required amounts. Also other compounds such as the indicator(s), stabilizers and/or antifolates may be added as a separate source, or optionally incorporated in the nutrient medium.

In a second embodiment of the third aspect, said kit comprises a thermostatic device, with the aid of which test samples can be kept at a pre-set temperature. The temperature may be one at which the microorganism shows sufficient growth. Preferably, said thermostatic device is designed in such a fashion that it can hold said containers filled with test medium. Optionally the thermostatic device is coupled to a means for setting the time needed for incubation such that heating and/or cooling is stopped after lapse of a pre-set period.

In a third embodiment of the third aspect of the invention, said kit comprises a data carrier loaded with a computer program suitable for instructing a computer to analyze digital data obtained from a sample-reading device. Said data carrier may be any carrier suitable for storing digital information such as a CD-ROM, a diskette, a DVD, a memory stick, a magnetic tape or the like. Advantageously, said data carrier loaded with a computer program also provides for easy access to the latest available computer programs suitable for use in the method of the present invention.

### Legend to the Figures

In FIG. 1 an overview is given of two embodiments of the device of the present invention (FIGS. 1A and 1B, respectively). Both embodiments comprise a first compartment (1) having a closed end (1.1) and an open end (1.2) and a second compartment (2) having two open ends (2.1 and 2.2). Inside first compartment (1), at closed end (1.1) a medium comprising a substance with which an analyte can be detected (1.3) may be present. Open end (2.2) has a means for withdrawing a predetermined volume of the fluid to be analyzed. In the embodiment of FIG. 1A said means is a space (2.3.1) formed by an insert (2.3.2) within second compartment (2). In the embodiment of FIG. 1B said means is an array of one or more volume markings (2.3.3) on the outside of the second compartment (2). Compartments (1) and (2) combined form the device of the present invention, i.e. compartment (0).

In FIG. 2 an overview is given of two embodiments of the device of the present invention (FIGS. 2A and 2B, respectively). Both embodiments comprise the features as already outlined for FIG. 1. Additionally, in FIG. 2 a third compartment (3) is attached to first compartment (1). Particles may be stored in third compartment (3), for instance in the form of a tablet. The material that forms the separation (3.1) between first compartment (1) and third compartment (3) is constructed of a material that can be broken when pressure is applied from the outside of third compartment (3) on its contents so that the contents can be brought inside of first compartment (1). Alternatively, third compartment (3) can be attached to second compartment (2).

In FIG. 3 an overview is given of an embodiment of the device of the present invention wherein first compartment (1) is shown in connection to second compartment (2). The connection is realized by member (4.1), which is in open position and may be folded in closed position (see FIG. 4). The shape of member (4.1) is of no particular importance, although it may be advantageous to use a rectangular shape, for instance when assembling several devices in a ‘tear-off’ matrix (see also FIG. 5). The thickness of member (4.1) may be any thickness, although it will be clear to the person skilled in the art that for the attachment of compartments (1) and (2) through apertures (4.2) and (4.3) respectively, a certain minimal thickness is required whereas a certain maximal thickness is needed when member (4.1) is to be folded, as is the case as illustrated in FIG. 4. Apertures (4.2) and (4.3) may be sealed with a removable material such as tape, plastic or a filmhinge. This material can advantageously be attached to the surface of member (4.1) in such a way that it covers apertures (4.2) and (4.3). Before sealing compartments (1) and/or (2), components that facilitate the detection of the analyte may be added to these compartments. For instance, for some types of microbial antibacterial tests, it may be advantageous to supply nutrients and/or indicators in the form of a granule, pill or tablet only moments before the actual analysis to the substance with which an analyte can be detected. Such a granule, pill or tablet can be added to compartment (2) before sealing. Obviously, in order to have an effective storage of said granule, pill or tablet, also the other open end (2.2) of compartment (2) should be closed. This is preferably performed using an easily removable capping system.

FIG. 4 is an illustration of the embodiment given in FIG. 3 in closed position. When the optionally present sealing of apertures (4.2) and (4.3) in the open position of FIG. 3 is removed, the device can be brought in the closed position and used after removal of an optionally present capping system. Advantageously, the sealing is chosen such that upon removal of the sealing adhesive material remains on the surface of member (4.1) so that upon folding the member along line (4.1.1) in the closed position of FIG. 4, parts (4.1.2) and (4.1.3) of member (4.1) will adhere to one another in a preferably liquid-tight and air-tight fashion.

FIG. 5 is an illustration of several devices according to FIG. 3 attached to one another. In FIG. 5, 12 devices are attached to one another in a 3×4 matrix, however also any other amount of devices in any other matrix is meant to be included in this illustration. Ideally, the devices are attached to one another using member (4.1) that allows for easy removal of individual devices, such as synthetic material equipped with perforated lines (4.1.4).

FIG. 6 is an illustration of an embodiment of the present invention wherein the device comprises compartments (1) and (2) and a removable lid (5). Furthermore, the device comprises an area of flexible material (6) suitable for temporary deformation. This area, optionally also present at the opposite site of the device, can be pressed with finger-pressure and is designed such that a predetermined amount of fluid is withdrawn when the pressure is released and open end (2.2)—after removal of the removable lid (5)—is placed in the fluid to be analyzed. Optionally, the devices of the present invention can also comprise a so-called drip-ring (7). This drip-ring retains small amounts of sample fluid that remain attached to the exterior of the device after insertion into the fluid to be analyzed and which may otherwise, upon placing the device in e.g. a heating device, may fall in the heating device. The device of FIG. 6 comprises a means for withdrawing a predetermined volume of fluid; said means being compartment 2 which can be filled with fluid using the area of flexible material (6) as outlined above.

FIG. 7 is an illustration of one possible way of preparing the devices of the present invention, more in particular the device as depicted in FIG. 6. Compartments (1) and (2) are constructed separately, i.e. by injection molding or blow molding, where after compartment (1) is partially filled with test medium. When the test medium has sufficiently solidified, compartments (1) and (2) are attached to one another to give compartment (0), for instance by gluing or, more preferably, by clicking together.

In FIG. 8, an illustration is given on the operation of the device of the present invention, more in particular the device as depicted in FIG. 6. In the first step (arrow A), the removable lid is removed, for instance by breaking and/or turning. Then the device is placed in the fluid to be analyzed (arrow B) and fluid is withdrawn by applying pressure as depicted by arrows C. When the fluid withdrawal is complete, the device is turned according to arrow D whereupon the fluid comes into contact with the test medium and the device can be placed in, for instance, an incubator.

FIG. 9 is an illustration of an embodiment of the present invention wherein the device comprises compartment (0), encompassing compartments (1) and (2) and a removable lid (5). Furthermore, the device comprises an area of flexible material (6) suitable for temporary deformation. This area, optionally also present at the opposite site of the device, can be pressed with finger-pressure and is designed such that a predetermined amount of fluid is withdrawn when the pressure is released and open end (2.2)-after removal of the removable lid (5)—is placed in the fluid to be analyzed. Also in FIG. 9 the optional drip-ring (7) is depicted. The device of FIG. 9 comprises a means (2.3) for withdrawing a predetermined volume of fluid; said means is an additional compartment (2.3.1) formed by an insert (2.3.2) within second compartment (2).

FIG. 10 is an illustration of one possible way of preparing the devices of the present invention, more in particular the device as depicted in FIG. 9. Compartments (1) and (2) are constructed preferably in one operation, i.e. by injection molding or blow molding, to form compartment (0) where after the device is partially filled with test medium through open end (2.2) along arrow E. Preferably the test medium is introduced in liquid form, for instance at elevated temperature, e.g. by means of an injection needle. Afterwards removable lid (5) can be attached using ordinary sealing techniques that are well known to the person skilled in the art.

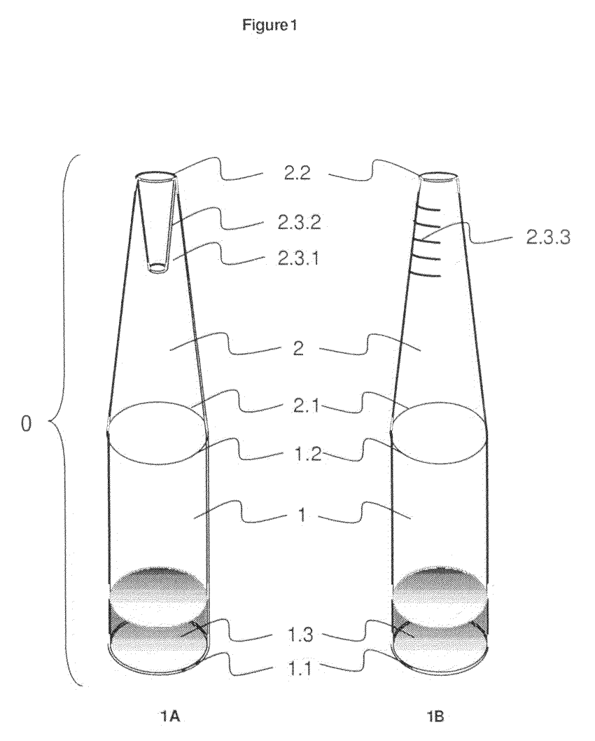

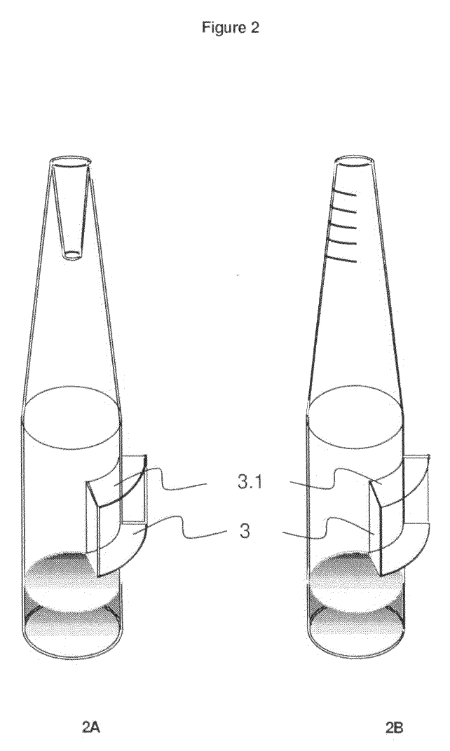

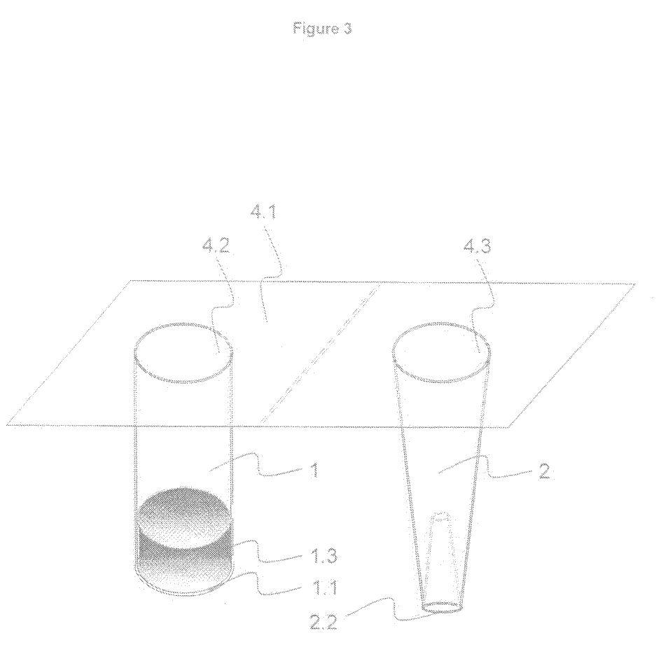

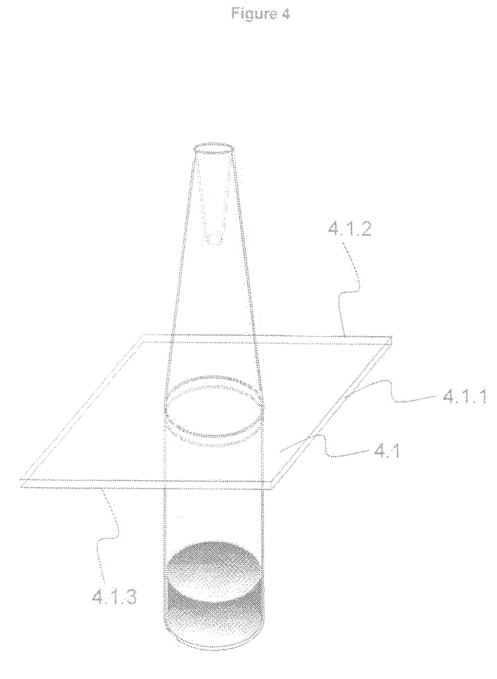

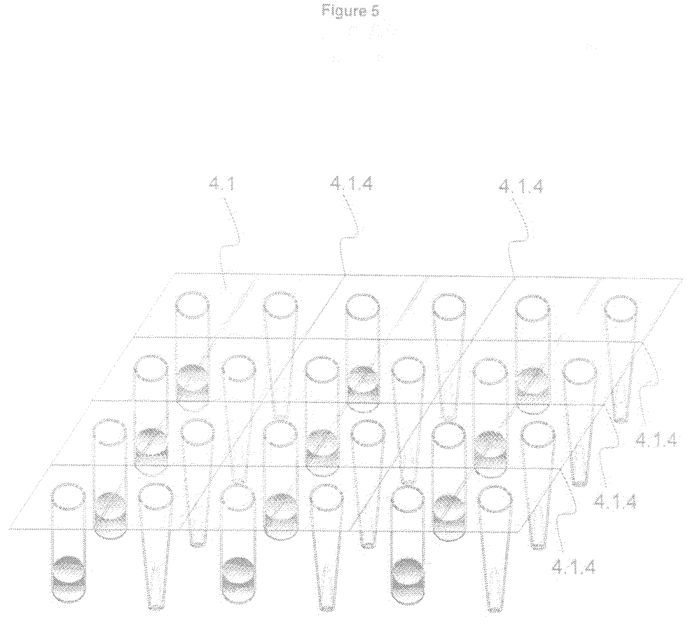

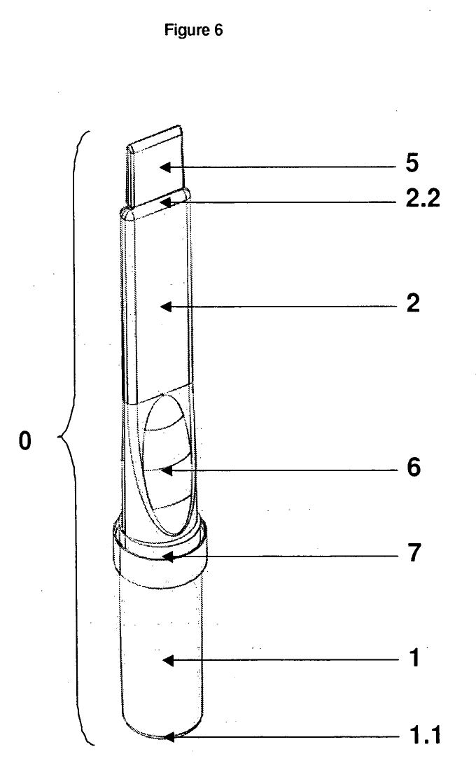

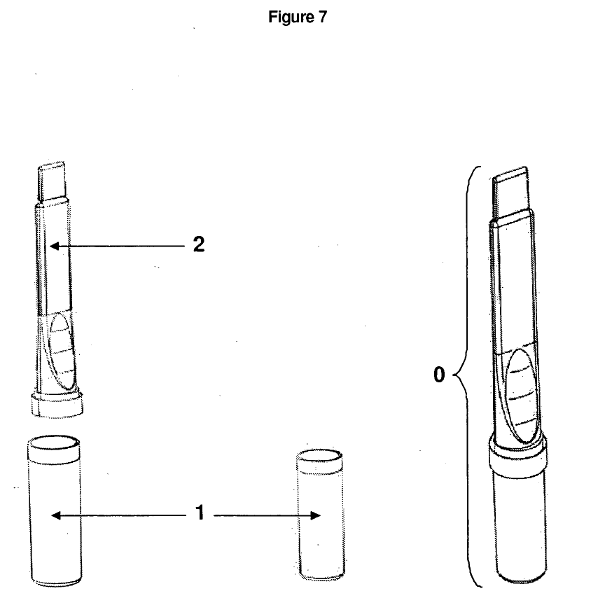

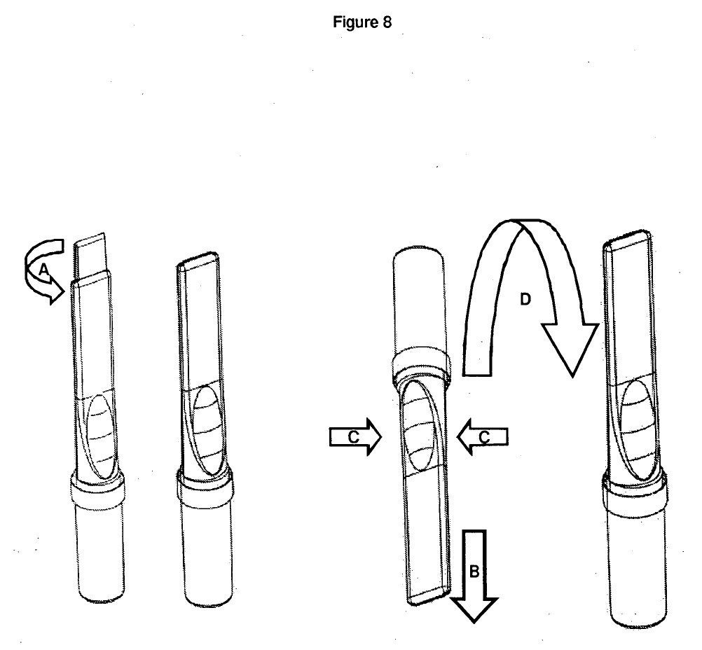

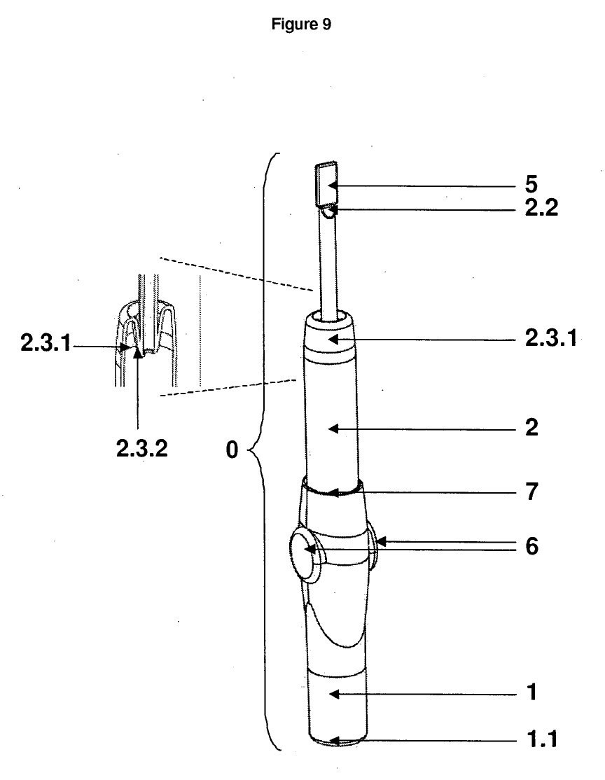

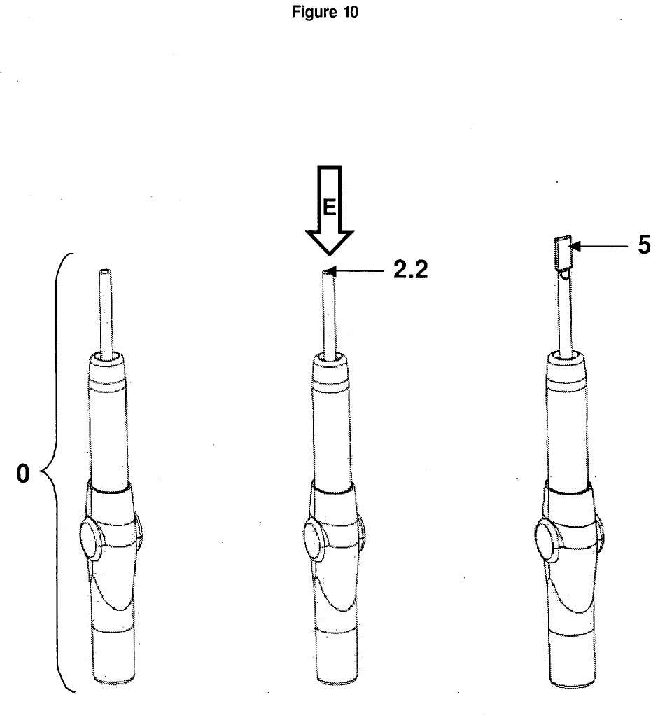

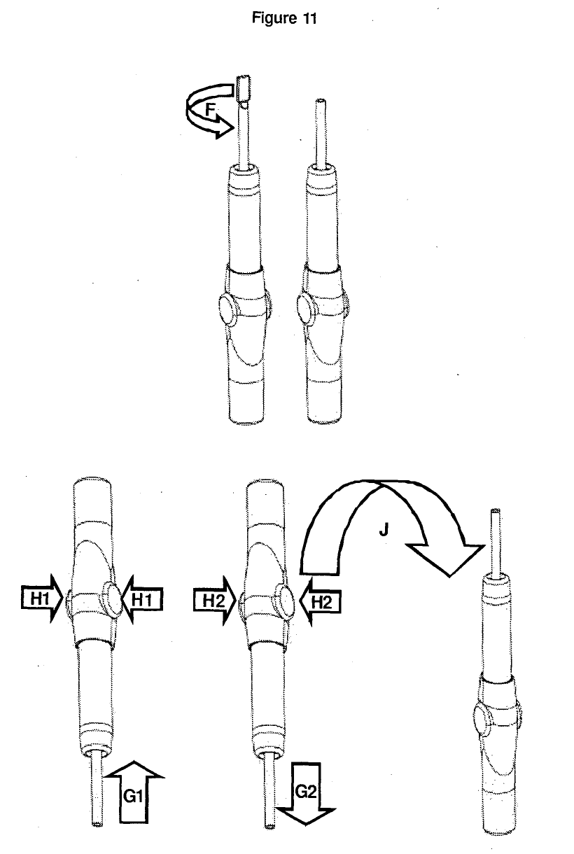

In FIG. 11, an illustration is given on the operation of the device of the present invention, more in particular the device as depicted in FIG. 9. In the first step (arrow F), the removable lid (5) is removed, for instance by breaking and/or turning. Then the device is placed in the fluid to be analyzed and fluid is withdrawn along arrow G1 by applying pressure as depicted by arrows H1. When the fluid withdrawal is complete, again pressure is applied along arrows H2, thereby removing excess fluid along arrow G2 whereby the required amount of fluid remains in the device in additional compartment 2.3.1. The device is then turned according to arrow J whereupon the fluid comes into contact with the test medium and the device can be placed in, for instance, an incubator.

1. Device for detecting an analyte in a fluid comprising a compartment (0) having a closed end (1.1) and an open end (2.2), wherein a test medium (1.3) comprising a substance with which said analyte can be detected is present in said compartment (0) at said closed end (1.1) and wherein said open end (2.2) has a means (2.3) for withdrawing a predetermined volume of said fluid.

2. Device according to claim 1 further comprising at least one area of flexible material (6) suitable for temporary deformation.

3. Device according to claim 1 further comprising a drip-ring (7).

4. Device according to claim 1 wherein said compartment (0) comprises a first compartment (1) having an open end (1.2) and a closed end (1.1) and a second compartment (2) having two open ends wherein the one open end (2.1) of the second compartment is connected to the open end (1.2) of the first compartment.

5. Device according to claim 4 wherein said open end (1.2) of said first compartment is connected to said open end (2.1) of said second compartment by a foldable member (4.1) comprising two apertures (4.2 and 4.3) whereto said first compartment and said second compartment are connected on the same side of said foldable member (4.1) and said apertures (4.2 and 4.3) on the opposite site of said foldable member (4.1) are placed adjacent to one another upon folding of said foldable member along a line perpendicular to, and intersecting in the middle of, a line connecting the centers of said two apertures.

6. Device according to claim 1 wherein said means for withdrawing a predetermined volume of fluid comprises an additional compartment (2.3.1).

7. Device according to claim 1 wherein said means for withdrawing a predetermined volume of said fluid comprises volume markings (2.3.3).

8. Device according to claim 1 further comprising a third compartment (3) wherein the interiors of compartment (0) and said third compartment (3) are separated by a material (3.1) suitable for being broken by the application of pressure.

9. Device according to claim 8 wherein said third compartment (3) contains a substance chosen from the list consisting of growth promoters, indicators, microorganisms, nutrients and sensitivity enhancers.

10. Device according to claim 1 wherein the open end (2.2) of said compartment (0) is closed with a removable lid (5).

11. A method for determining the presence or absence of an analyte in a fluid comprising:
(f) inserting the open end (2.2) of said compartment (0) of the device of any one of claims 1 to 9 into said fluid;
(g) optionally applying external pressure on said compartment (0) followed by release of pressure;
(h) withdrawing said device from said fluid;
(i) optionally removing excess fluid from the interior of said device until the required volume is obtained;
(j) turning said device such that the fluid remaining in said device is contacted with a test medium (1.3) suitable for detecting said analyte.

12. Method according to claim 11 wherein said analyte is an antibiotic and said test medium comprises a test microorganism and at least one indicator.

13. Method according to claim 12, further comprising:
(k) incubating said test microorganism with the fluid under conditions whereby growth of the test microorganism occurs if no antibiotic is present in the fluid sample; and
(l) detecting any growth or inhibition of growth of the test microorganism as appropriate by means of an indicator,

14. Use of a device according to claim 1 for sampling and analyzing a fluid.
